(19) United States
(12) Patent Application Publication
(10) Pub. No.: US 2010/0193433 A1
(43) Pub. Date: Aug. 5, 2010

(54) Vertical Land-Based Bioreactor

(75) Inventors: Volker A. Hausin, Rochester, NY (US); Daniel J. LaGassee, Lyons, NY (US)

Correspondence Address: BOND, SCHOENECK & KING, PLLC, ONE LINCOLN CENTER, SYRACUSE, NY, 13202-1355, US

(73) Assignee: TVT US CORP., Rochester, NY (US)

(21) Appl. No.: 12/602,355

(22) PCT Filed: May 23, 2008
(86) PCT No.: PCT/US08/64615

§ 371 Date: Nov. 30, 2009

Related U.S. Application Data
(60) Provisional application No. 60/941,272, filed on May 31, 2007.

(51) Int. Cl. C02F 3/02; C12M 3/00

(52) U.S. Cl. 210/620; 435/296.1; 210/601; 210/150

(57) ABSTRACT

A bioreactor for treating a liquid comprises a container having a vertical wall portion with at least one inlet port and at least one outlet port. The bioreactor also comprises a media support platform securely disposed inside the container at a position below the inlet and outlet ports, and substantially permeable to liquid. The bioreactor comprises at least one cylindrical chamber inside the container at a position above the media support platform. The bioreactor also comprises a plurality of media units topically disposed on the media support platform. The bioreactor comprises a recirculation/aeration system secured at a bottom end of the cylindrical chamber. The recirculation/aeration system has a submersible pump device that can be programmed to pump liquid at variable speeds and has a liquid intake portion having perforations, an air intake tube, and an eductor device. The air intake tube protrudes through a top port of the container.

### RELATED APPLICATIONS

This application claims priority to U.S. provisional patent application 60/941,272 entitled VERTICAL LAND BASED BIO-REACTOR which was filed on May 31, 2007.

### FIELD

The claimed invention relates to a vertical land-based bioreactor for treating liquids with a bioactive microorganism.

### BACKGROUND

Over the years, various methods and devices have been developed to treat liquids from various sources (e.g., wineries, farms, dairy plants, sewage treatment plants). One example relates to wastewater treatment processes that use biological microorganisms to remove unwanted materials from the wastewater (see, e.g., U.S. Pat. No. 5,228,998). However, current methods of treating liquids in bioreactors using microorganisms can be inefficient. One of the problems with using microorganisms in bioreactors relates to the inefficiencies of loading and unloading media into the bioreactor. Another problem relates to the maintenance required for cleaning the interior portion of the bioreactor, as well as maintaining the various components contained in the bioreactor. Therefore, it is desirable to have a bioreactor apparatus, a system containing the bioreactor apparatus, and a method for using the bioreactor apparatus for treating liquids with bioactive microorganisms in an efficient, cost-effective, and environmentally friendly way. Further, it is desirable to be able to load and unload media (e.g., substrates for biofilm growth) in an efficient way. It is also desirable to be able to retrofit existing land-based containers to convert them into bioreactors for treating liquids using microorganisms. The present invention is directed to addressing these and other deficiencies in the field of liquid treatment using bioreactors and microorganisms.

### SUMMARY

A bioreactor for treating a liquid is disclosed. The bioreactor has a container comprising a top portion, a vertical wall portion, and a bottom portion, wherein the top portion comprises at least a top port that is either sealed or unsealed, and wherein the vertical wall portion comprises at least one inlet port and at least one outlet port. The bioreactor also has a media support platform securely disposed inside the container at a position below the inlet and outlet ports, wherein the media support platform is substantially perpendicular to the container's vertical wall portion and substantially spans the cross-sectional area of the container, and wherein the media support platform is permeable to liquid. The bioreactor further has at least one cylindrical chamber securely disposed inside the container at a position above the media support platform, wherein the at least one cylindrical chamber comprises a top end, a bottom end, and a sidewall portion. The bioreactor also has a plurality of media units topically disposed on the media support platform in an outer chamber portion, wherein the outer chamber portion is defined by the space between the sidewall portion of the at least one cylindrical chamber and the vertical wall portion of the container. The bioreactor further has a recirculation/aeration system secured at the bottom end of the cylindrical chamber, wherein the recirculation/aeration system comprises a submersible pump device that can be programmed to pump liquid at variable speeds and that comprises a liquid intake portion having perforations, an air intake tube, and an eductor device, and wherein the air intake tube protrudes through the top port of the container's top portion.

A method for treating a liquid is disclosed. A bioreactor is provided. The provided bioreactor has a container comprising a top portion, a vertical wall portion, and a bottom portion, wherein the top portion comprises at least a top port that is either sealed or unsealed, and wherein the vertical wall portion comprises at least one inlet port and at least one outlet port. The provided bioreactor also has a media support platform securely disposed inside the container at a position below the inlet and outlet ports, wherein the media support platform is substantially perpendicular to the container's vertical wall portion and substantially spans the cross-sectional area of the container, and wherein the media support platform is permeable to liquid. The provided bioreactor further has at least one cylindrical chamber securely disposed inside the container at a position above the media support platform, wherein the at least one cylindrical chamber comprises a top end, a bottom end, and a sidewall portion. The provided bioreactor also has a plurality of media units topically disposed on the media support platform in an outer chamber portion, wherein the outer chamber portion is defined by the space between the sidewall portion of the at least one cylindrical chamber and the vertical wall portion of the container. The provided bioreactor further has a recirculation/aeration system secured at the bottom end of the cylindrical chamber, wherein the recirculation/aeration system comprises a submersible pump device that can be programmed to pump liquid at variable speeds and that comprises a liquid intake portion having perforations, an air intake tube, and an eductor device, and wherein the air intake tube protrudes through the top port of the container's top portion. An untreated liquid is imported into the bioreactor. The untreated liquid is recirculated within the bioreactor to produce a treated liquid. The treated liquid is exported from the bioreactor.

A system for treating a liquid is also disclosed. The system has a source of untreated liquid. The system also has at least one bioreactor. The system's at least one bioreactor has a container comprising a top portion, a vertical wall portion, and a bottom portion, wherein the top portion comprises at least a top port that is either sealed or unsealed, and wherein the vertical wall portion comprises at least one inlet port and at least one outlet port. The system's at least one bioreactor also has a media support platform securely disposed inside the container at a position below the inlet and outlet ports, wherein the media support platform is substantially perpendicular to the container's vertical wall portion and substantially spans the cross-sectional area of the container, and wherein the media support platform is permeable to liquid. The system's at least one bioreactor further has at least one cylindrical chamber securely disposed inside the container at a position above the media support platform, wherein the at least one cylindrical chamber comprises a top end, a bottom end, and a sidewall portion. The system's at least one bioreactor also has a plurality of media units topically disposed on the media support platform in an outer chamber portion, wherein the outer chamber portion is defined by the space between the sidewall portion of the at least one cylindrical chamber and the vertical wall portion of the container. The system's at least one bioreactor further has a recirculation/aeration system secured at the bottom end of the cylindrical chamber, wherein the recirculation/aeration system comprises a submersible pump device that can be programmed to pump liquid at variable speeds and that comprises a liquid intake portion having perforations, an air intake tube, and an eductor device, and wherein the air intake tube protrudes through the top port of the container's top portion. The at least one bioreactor is coupled to the source of untreated liquid and configured to receive untreated liquid from the source and to treat the untreated liquid to produce treated liquid. The system also has a collection unit for collecting the treated liquid from the at least one bioreactor, wherein the collection unit is coupled to the at least one bioreactor, and wherein said collection unit is configured to clarify and/or agitate the collected treated liquid.

A kit for converting a container into a bioreactor for treating a liquid is further disclosed. The kit has a plurality of media support panels that combine to form a media support platform inside a container, wherein the media support platform is permeable to liquid. The kit also has at least one cylindrical chamber comprising a top end, a bottom end, and a sidewall portion, wherein the bottom end is configured to be secured inside the container. The kit further has a plurality of media units contained in at least one flexible mesh encasement. The kit also has a recirculation/aeration system comprising a submersible pump device that can be programmed to pump liquid at variable speeds and that comprises a liquid intake portion having perforations, an air intake tube, and an eductor device.

A method for converting a container into a bioreactor for treating a liquid is further disclosed. A container is provided. The container has a top portion, a vertical wall portion, and a bottom portion. The top portion has at least a top port that is either sealed or unsealed. The vertical wall portion has at least one inlet port and at least one outlet port. A media support platform is inserted inside the container at a position below the inlet and outlet ports, wherein the media support platform is substantially perpendicular to the container's vertical wall portion and substantially spans the cross-sectional area of the container. The media support platform is permeable to liquid. At least one cylindrical chamber is deposited inside the container at a position above the media support platform. The at least one cylindrical chamber has a top end, a bottom end, and a sidewall portion. At least one flexible mesh encasement is loaded onto the media support platform in an outer chamber portion of the container. The outer chamber portion is defined by the space between the sidewall portion of the at least one cylindrical chamber and the vertical wall portion of the container. The at least one flexible mesh encasement has a plurality of media units. A recirculation/aeration system is installed inside the cylindrical chamber. The recirculation/aeration system has a submersible pump device that can be programmed to pump liquid at variable speeds and that comprises a liquid intake portion having perforations, an air intake tube, and an eductor device. The air intake tube protrudes through the top port of the container's top portion.

A method of unloading media units from a container is also disclosed. A container having a plurality of flexible mesh encasements each containing a plurality of media units and each having two opposed ends is identified. The plurality of flexible mesh encasements are serially linked together from end to end to form a chain of encasements, wherein said chain comprises a first end encasement and an opposing second end encasement. The chain of encasements is extracted from the container, thereby unloading the plurality of media units from the container.

Further objects and advantages of the claimed invention will become more apparent in the detailed description of the preferred embodiment and other embodiments presented below.

### BRIEF DESCRIPTION OF THE DRAWINGS

FIG. 1 is a schematic illustration of a longitudinal section through one embodiment of a bioreactor for treating a liquid using bioactive microorganisms.

FIG. 2 is a schematic of illustrative examples of mechanisms for attaching a cylindrical chamber to a media support platform of the bioreactor of the present invention.

FIGS. 3A-3E show several embodiments of the mesh encasement and media units of the present invention.

FIG. 4 is a schematic illustration of a longitudinal section through one embodiment of a bioreactor of the present invention.

FIG. 5 is a schematic illustration of a longitudinal section through one embodiment of a bioreactor of the present invention.

FIG. 6 is a schematic illustration of a longitudinal section through one embodiment of a bioreactor of the present invention.

FIG. 7 is a schematic illustration of one embodiment of a system for treating liquid of the present invention.

FIG. 8 is a schematic illustration of one embodiment of a system for treating liquid of the present invention.

FIG. 9 is a schematic illustration of one embodiment of a media support platform of the present invention and one embodiment of the plurality of grating units that combine to form the media support platform.

FIGS. 10A-10B illustrate various embodiments of the chain of mesh encasements of the present invention.

FIGS. 11A-11C illustrate various embodiments of the cylindrical chambers 40 as arranged in the container 20. The vantage point of FIGS. 11A-11C is from a top view of a cross-section of a bioreactor of the present invention.

It will be appreciated that for purposes of clarity and where deemed appropriate, reference numerals have been repeated in the figures to indicate corresponding features, and that the various elements in the drawings have not necessarily been drawn to scale in order to better show the features.

### DETAILED DESCRIPTION

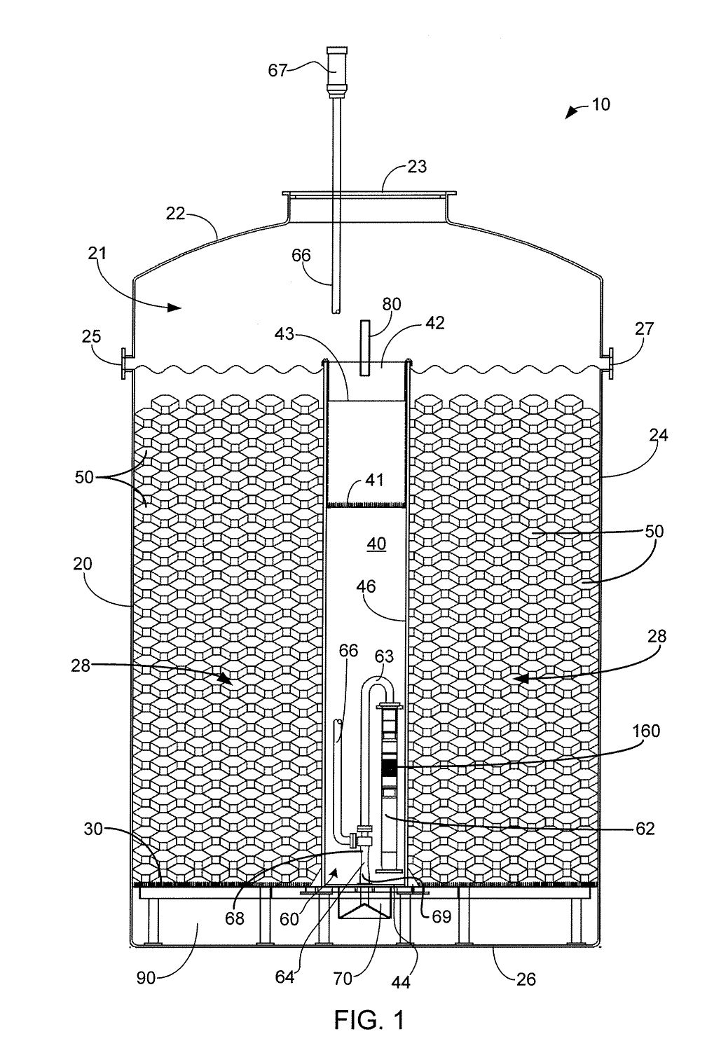

FIG. 1 illustrates one embodiment of a bioreactor 10 for treating a liquid using bioactive microorganisms. In this embodiment, the bioreactor 10 includes a container 20 having the following interior components: a media support platform 30, a cylindrical chamber 40, a plurality of media units 50, and a recirculation/aeration system 60.

The container 20 has a top portion 22, a vertical wall portion 24, and a bottom portion 26. The top portion 22 includes a top port 23, which is an opening of sufficient size to allow for insertion of the above-listed interior components of the bioreactor 10, where the interior components can be either partially or fully assembled prior to insertion into the interior portion 21 of the container 20. The vertical wall portion 24 of the container 20 includes at least one inlet port 25 and at least one outlet port 27. In the particular embodiment of FIG. 1, the inlet port 25 is located substantially opposite to the outlet port 27, although they need not be substantially opposite one another. The inlet port 25 is used for importing untreated liquid into the interior portion 21 of the container 20. The outlet port 25 is used to export treated liquid out of the interior portion 21 of the container 20. The container 20 can be made of any material suitable for holding liquids. Examples of such materials, include, but are not limited to, high density polyethylene (“HDPE”), stainless steel, concrete, and the like. The shape of the container can be any shape, as described more fully below.

The media support platform 30 is securely disposed inside the container 20 at a position below the inlet port 25 and the outlet port 27, but above the bottom portion 26 of the container 20. The media support platform 30 includes a plurality of media support panels that fit together to make up the fully assembled media support platform 30. The separate media support panels need not be of the same size or shape. The media support platform 30 can be made of various synthetic materials, including, for example, fiberglass and stainless steel. When fully assembled, the media support platform 30 is permeable to liquid, yet strong enough to support the combined weight of the plurality of media units 50, the cylindrical chamber 40, and the recirculation/aeration system 60. Further, the fully assembled media support platform 30 substantially spans the cross-sectional area of the interior portion of the container 20.

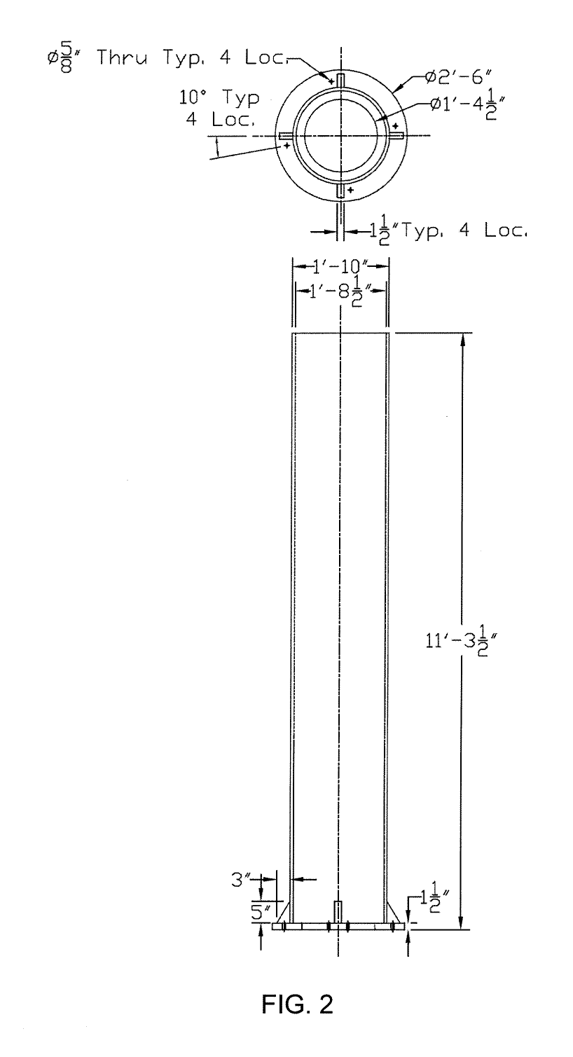

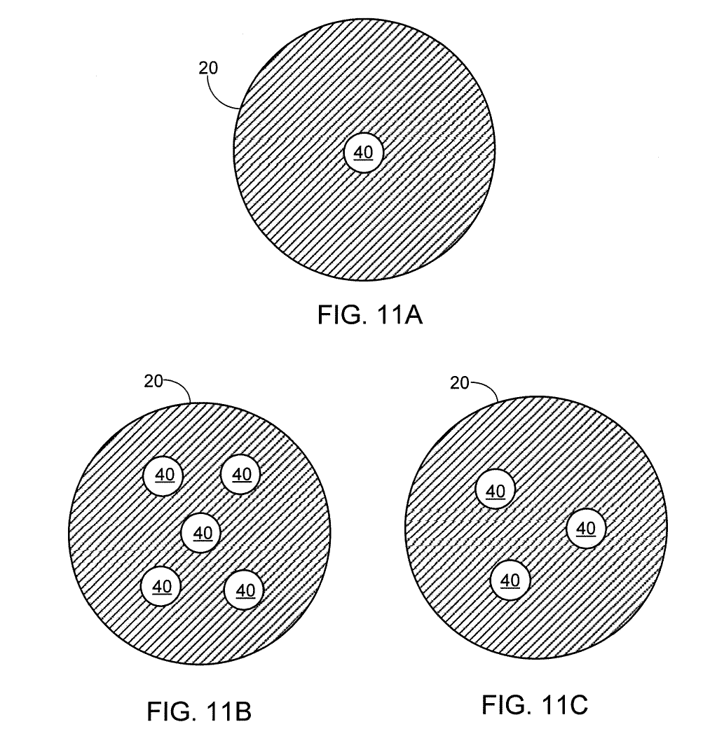

The cylindrical chamber 40 is a hollow tube-like structure having a top end 42 (which is fully open or partially open to allow liquid to enter the cylindrical chamber 40), a bottom end 44 (which is completely or substantially closed so that it is not permeable to liquid except through the submersible pump device 62, described below), and a sidewall portion 46. The cylindrical chamber 40 is positioned in the interior portion 21 of the container 20 at a position that is substantially directly below the top port 23 of the top portion 22 of the container 20. However, in other embodiments, the cylindrical chamber 40 need not be substantially below the top port 23. Further, a plurality of cylindrical chambers 40 can be included in the container (see, e.g., FIGS. 11A-C, which shows a top view of a cross-section of a bioreactor of the present invention). In one embodiment, the cylindrical chamber 40 can be independently supported and secured in place by means independent of the media support platform 30. For example, the cylindrical chamber 40 can be secured to the bottom portion 26 of the container 20. The bottom end 44 of the cylindrical chamber 40 can alternatively be secured to the media support platform 30. FIG. 2 further illustrates various mechanisms for securing the cylindrical chamber 40 to the media support platform 30. The cylindrical chamber 40 can be made of any material that is impermeable to liquid, including, but not limited to, HDPE and the like. In the embodiment of FIG. 1, the top end 42 is substantially as high as the inlet port 25 and outlet port 27. However, in other embodiments, the top end 42 of the cylindrical chamber 40 can be of variable height, either below or above the inlet port 25 and the outlet port 27.

Once in place, the at least one cylindrical chamber 40 and the media support platform 30 combine with the vertical wall portion 24 of the container 20 to final an outer chamber portion 28, which is defined by the space between the sidewall portion 46 of the at least one cylindrical chamber 40 and the vertical wall portion 24 of the container 20, and extending upward from the media support platform 30 to the top end 42 of the cylindrical chamber 40.

The plurality of media units 50 are contained within the outer chamber portion 28 (as described above). For illustrative purposes, the particular embodiment illustrated in FIG. 1 shows the plurality of media units 50 filling the outer chamber portion 28 up to the point that is proximate to the top end 42 of the cylindrical chamber 40. However, the plurality of media units 50 can be filled at any height, up to the top end 42 of cylindrical chamber 40. The media units 50 can be made of any material that promotes biofilm growth on the media units 50. Suitable examples of media units 50 can include, but are not limited to, polyethylene or polypropylene packing media (e.g., Q-PAC), or the like. The media units 50 are used to assist in optimizing the growth of any bioactive microorganisms added to the bioreactor 10. See, e.g., U.S. Pat. No. 5,228,998, which is hereby incorporated by reference in its entirety. In particular, the media units 50 support the growth of at least one type of biological microorganism. Thus, it may be desirable in some embodiments to introduce at least one type of microorganism into the bioreactor 10. The media units 50 can serve as a food source to the microorganisms and can sustain much larger colonies of microorganisms (e.g., microbacteria), and at higher growth rates, than if the microorganisms had to find the food. There are many naturally occurring microorganisms which have been procured and stored in the American Type Culture Collection. Those skilled in the art know that they can go to the American Type Culture Collection, look-up a microorganism which is known to feed on the type of waste desired (see, e.g., U.S. Pat. No. 4,925,564, which is hereby incorporated by reference in its entirety), and can purchase a supply of the microorganisms for dispersal within the bioreactor 10.

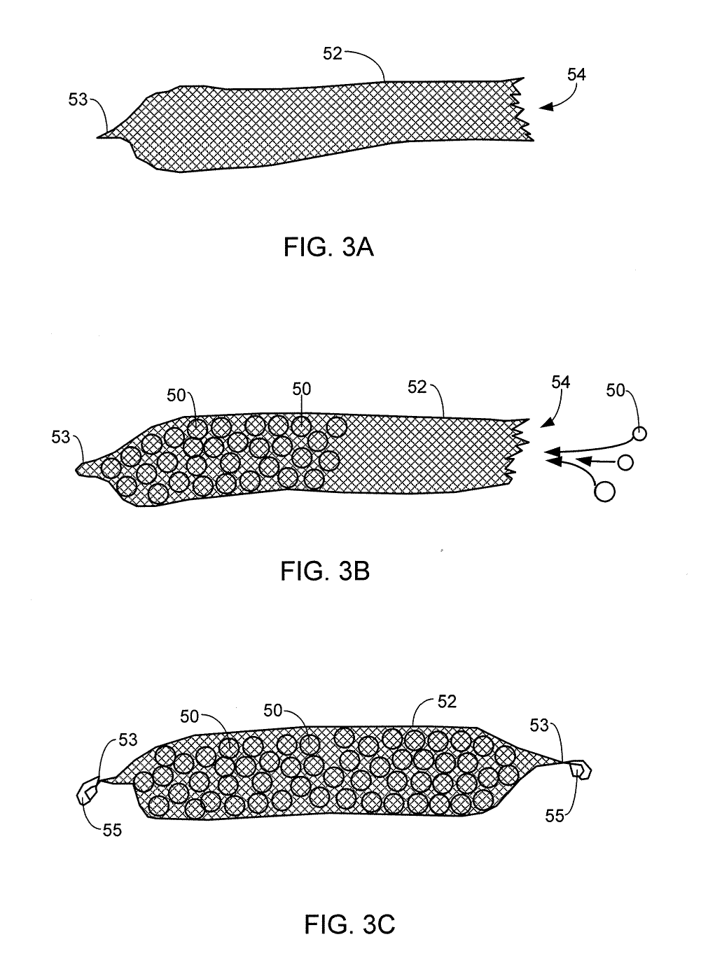

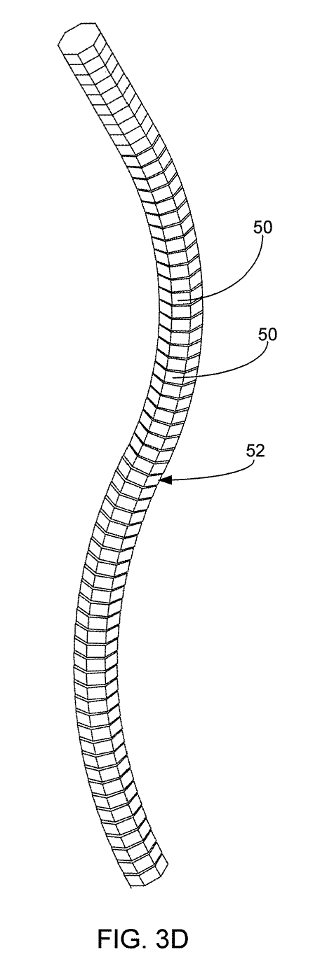

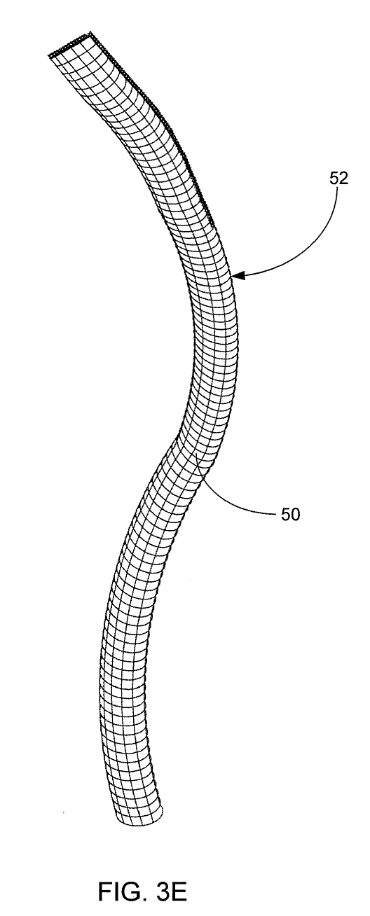

As shown in more detail in FIGS. 3A-3E, the plurality of media units 50 can be contained in a plurality of mesh encasements 52, which allows for the plurality of media units 50 to be efficiently loaded into and unloaded out of the outer chamber portion 28. FIG. 3A illustrates an unfilled mesh encasement 52 having a closed end 53 and an open end 54. FIG. 3B illustrates a partially filled mesh encasement 52 having a closed end 53, and an open end 54 through which media units 50 can be inserted. Once the mesh encasement 52 is filled with a desirable amount of media units 50, the open end 54 can be closed. Further, as shown in FIG. 3C, in one embodiment, the hooking structures 55 (e.g., carabiner-type clips or hooks) can be attached to one or both ends of the mesh encasement 52 in order to facilitate loading and unloading of the plurality of media units 50 contained within each mesh encasement 52. FIG. 3D and FIG. 3E show further embodiments of a filled mesh encasement. Below is a further discussion of the mesh encasements and how a plurality of these encasements can be linked together to form a chain, and how such a configuration can aid in efficient loading and unloading of the encasements from the container.

The recirculation/aeration system 60 is substantially housed within the cylindrical chamber 40 and includes a submersible pump device 62, an eductor device 64, and an air intake tube 66. The air intake tube 66 is connected at one end to the eductor device 64 and extends upward through the cylindrical chamber 40 and protrudes through the top port 23, and terminates at an opposite end 67 located outside the container 20. For clarity, the air intake tube 66 is shown in broken form to facilitate viewing of intervening detail. The eductor device 64 has a connection end 68 and an export end 69. The submersible pump device 62 is connected to the connection end 68 of the eductor device 64 with a connecting tube 63. The eductor device 64 is secured to the bottom end 44 of the cylindrical chamber 40, thereby securing the recirculation/aeration system 60 in place. A suitable eductor device 64 includes, for example, a venturi-type eductor device. The submersible pump device 62 includes a liquid intake portion 160 that collects liquid from the cylindrical chamber 40 and pumps the liquid through the eductor device 64 so that the liquid exits the cylindrical chamber 40 through the export end 69 of the eductor device 64. The liquid intake portion 160 also includes perforations through which the liquid can flow. A suitable submersible pump device 62 is one that can pump liquid at variable speeds or at constant speeds, and that can be programmed to pump continuously or intermittently at a constant or variable speed. The export end 69 can be associated with a flow adapter 70 (also referred to herein as a diverter for directing hydraulic flow) to promote even flow distribution of the liquid from the eductor device 64. FIG. 1 shows the flow adapter 70 as having an inverted cone structure, although the present invention is not limited to such structure and can have other structures (e.g., a 4-sided pyramid structure).

FIG. 1 further illustrates additional aspects of one embodiment of the bioreactor 10 of the present invention. For example, the cylindrical chamber 40 can contain a maintenance platform 41 that is horizontally disposed within the cylindrical chamber 40 at a position above the submersible pump device 62. A suitable maintenance platform 41 is one that is permeable to liquid and configured to support the weight of a person (e.g., a weight of at least 200 pounds). The maintenance platform 41 can be used to support a person while that person inserts or exports the plurality of media units 50, or while that person otherwise inspects or maintains the interior portion of the container 20 or the components contained therein. A particular type of maintenance platform 41 can be associated with a basket-like structure such that the bottom of the basket-like structure is the maintenance platform 41 and the vertical side of the basket-like structure extends upward and connects at the top end 42 of the cylindrical chamber 40. The cylindrical chamber 40 can also include a catch screen 43 that is configured to prevent solid material of a desired size from entering the cylindrical chamber 40 and reaching the submersible pump device 62. The catch screen 43 should be configured so that it prevents solid materials of a size larger than the perforations of the liquid intake portion of the submersible pump device from reaching the liquid intake portion 160. The catch screen 43 is horizontally secured inside the cylindrical chamber 40 at a position above the submersible pump device, and more particularly above the maintenance platform 41, and can be associated with a basket-like structure such that the bottom of the basket-like structure is the catch screen 43 and the side of the basket-like structure extends upward and connects at the top end 42 of the cylindrical chamber 40. The catch screen can also be at the top end 42 of the cylindrical chamber 40.

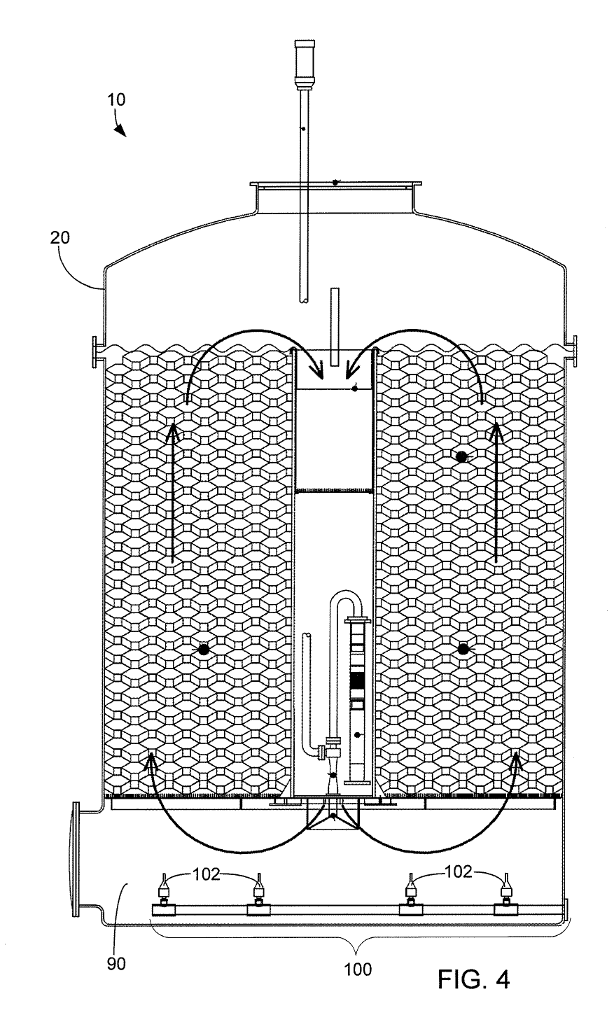

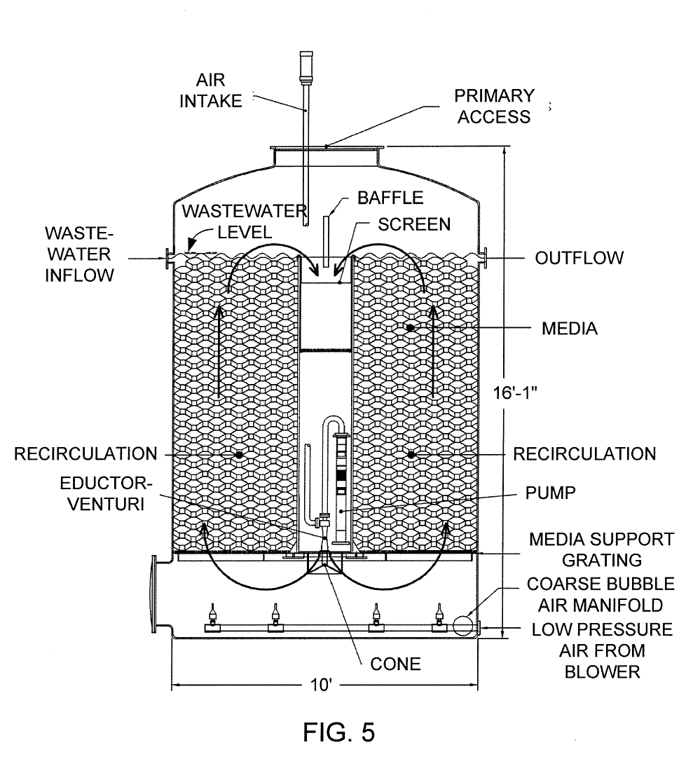

The bioreactor 10 can also include a baffle 80 for directing surface flow of liquid within the container 20. In the embodiment illustrated in FIG. 1, the baffle 80 is positioned at the top end 42 of the cylindrical chamber 40 to promote flow of surface liquid into the top end 42. Other types of suitable baffle arrangements are illustrated in FIGS. 4 and 5 (described below).

As illustrated in FIG. 4, the bioreactor 10 can also include an air supply device 100 located in the bottom chamber 90 of the container 20. The air supply device 100 can include a plurality of nozzles 102 through which air or other gaseous mixtures can be introduced into liquid contained in the bottom chamber 90. Introduction of the air or other gaseous mixtures into the bottom chamber 90 can result in coarse bubble diffusion to prevent undesirable buildup of debris. Different types of microorganisms utilize different types of energy sources (e.g., food substance) and will thrive in relation to the food substance that is available, provided optimal conditions for growing are present, such as a large surface area to grow on and generous amounts of oxygen for aerobic bacteria. Therefore, the air supply device 100 can be used to provide oxygen to the microorganisms.

Furthermore, the bioreactor can include an agitator which assists with the recirculation of liquid upward through the plurality of media units 50 contained in the outer chamber portion 28. Some suitable examples of agitators include, but are not limited to, a propeller, an impeller, and a water jet.

The present invention further relates to a method for treating a liquid (e.g., by using microorganisms). To illustrate one embodiment of this method, reference can be made to the bioreactor illustrated in FIG. 1. In one embodiment, the method involves providing bioreactor 10. The untreated liquid is imported into the container 20 of bioreactor 10, and more particularly into the inlet port 25. Importing the untreated liquid into container 20 can be achieved by making use of gravity or by using a pumping device for such purpose, as is well known by those of ordinary skill in the art. Importation of the untreated liquid can be adjusted and maintained so as to achieve a desirable flow rate of the untreated liquid into the container 20.

Once the untreated liquid is in the interior portion 21 of the container 20, the untreated liquid is recirculated within the bioreactor 10 to produce a treated liquid. In one embodiment, recirculation includes flowing the untreated liquid through at least one flow cycle. As used in this embodiment, one flow cycle includes running the untreated liquid through the recirculation/aeration system 60 to produced aerated untreated liquid and then directing the aerated untreated liquid upward through the media units 50 contained in the outer chamber portion 28. The recirculation step can include one or more flow cycles to produce a treated liquid. The treated liquid can then be exported from the bioreactor 10 through the outlet port 27 of the container 20.

In one embodiment of the method for treating microorganisms, the top portion 22 can remain substantially open. This embodiment will allow for natural release of gases from the interior portion 21 of the container 20. In another embodiment, the top port 21 can be sealed using a top cover, which allows for gases to build within the container 20. The top cover can be configured to have a release valve to allow for controlled release of gases from the container 20.

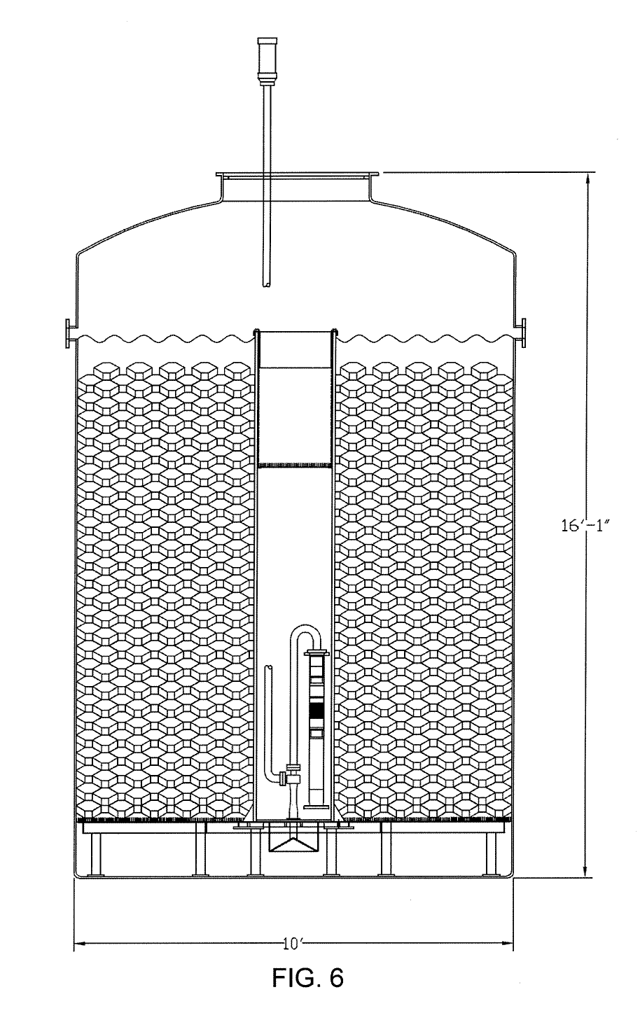

As illustrated in FIGS. 1, 5, and 6, liquid can enter the cylindrical chamber 40 at the top end 42 and flow downward toward the submersible pump device 62. If catch screen 43 is in place, any particulate matter contained in the liquid that is larger in size than the openings of the catch screen 43 will be trapped and prevented from flowing down toward the submersible pump device 62. Once the liquid reaches the submersible pump device 62, the recirculation/aeration system 60 works to pump the liquid through the eductor device 64 and exit the export end 69 into the bottom chamber 90. A flow adapter 70 can be attached to the export end 69 to facilitate even distribution of the liquid from the export end 69. Thereafter, the liquid then flows or percolates upward through the media units 50 until the liquid reaches the surface region. During the recirculation of the liquid, the microorganism contained in the media units 50 can work on the liquid to produce a treated liquid that is ready for export through the outlet port 27.

Introduction of at least one type of microorganism into the bioreactor is contemplated by the method of treating liquid of in the bioreactor of the present invention. Introduction of microorganisms can be done prior to importation of any liquid into the bioreactor, during the importation of the liquid into the bioreactor, or after importation of the liquid at a point when no further liquid is being imported into the bioreactor. Introduction is effected by contacting the at least one type of microorganism to the media units contained in the bioreactor. Suitable amounts (concentrations) and types of microorganisms can be determined according to the desired treatment regimen for a particular type of untreated liquid.

The method can further include promoting coarse bubble diffusion of air or any other suitable gas or gas mixture upwardly from the bottom chamber 90 through the media units 50.

The present invention also relates to a bioreactor system that includes at least one source of untreated liquid, at least one bioreactor for treating the untreated liquid, and at least one collection unit for collecting the treated liquid from the at least one bioreactor. As used herein, the term “untreated liquid” can include any liquid that is treatable using bioactive microorganisms. Therefore, the source of untreated liquid can be any body of water or fluid containment area that contains untreated liquid. The collection unit can be configured to clarify and/or agitate the collected treated liquid.

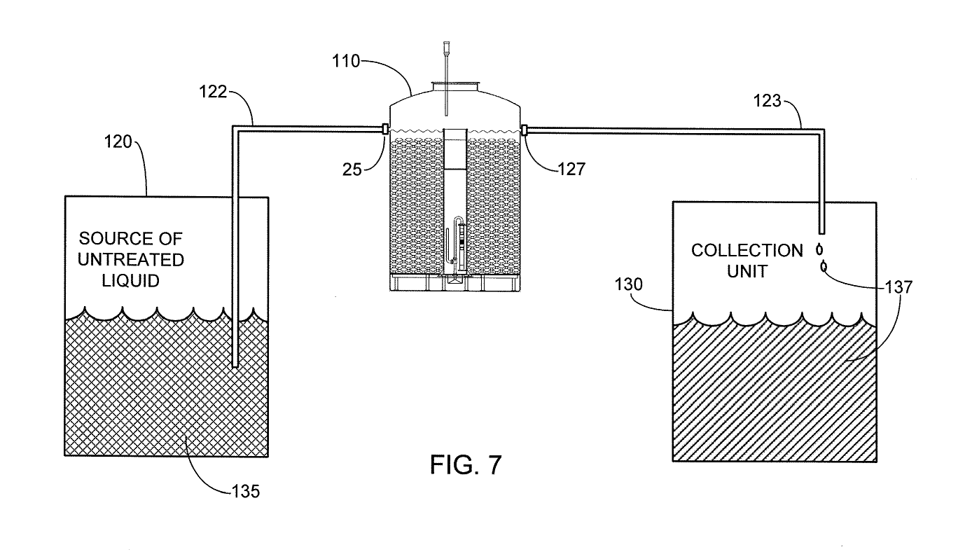

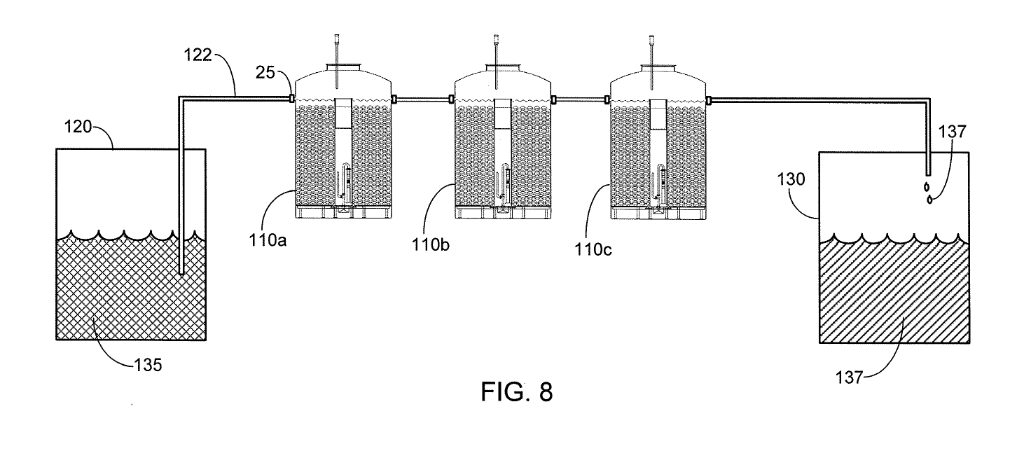

FIGS. 7-8 illustrate alternative embodiments of the bioreactor system of the present invention.

FIG. 7 illustrates an embodiment of a bioreactor system that includes one source 120 of untreated liquid 135, one bioreactor 110, and one collection unit 130. Source 120 contains untreated liquid. The untreated liquid is transported from source 120 to the bioreactor 110 through import tube 122, which connects to inlet port 25 of the bioreactor 110. Transportation of the untreated liquid 135 into the bioreactor 110 can be through gravity flow or can be facilitated using a pumping device. Suitable pumping devices for such use are well known in the art. The untreated liquid is deposited into the bioreactor 110 for treatment. The untreated liquid is treated by the bioreactor 110 using a batch process, a continuous flow process, or a hybrid batch/continuous flow process, as described more fully herein. After treatment, the treated liquid 137 is then exported out of the bioreactor 110 through the outlet port 27, whereby the treated liquid travels through an export tube 123 and collects in the collection unit 130.

FIG. 8 illustrates an embodiment of a bioreactor system that includes a plurality of bioreactors serially linked together. The untreated liquid 135 is transported from source 120 to the bioreactor 110a through import tube 122, which connects to inlet port 25 of the bioreactor 110a. After being treated in bioreactor 110a, the liquid can be exported from bioreactor 110a and imported into bioreactor 110b for further treatment. After being treated in bioreactor 110b, the liquid can be exported from bioreactor 110b and imported into bioreactor 110c. The treated liquid 137 can then be collected in collection unit 130. In other embodiments, additional bioreactors can be serially linked together between the source 120 and the collection unit 130, as desired.

The present invention also relates to a kit for converting a container having an open top port into a land-based bioreactor for treating a liquid. This kit can be used for converting any land-based container into a land-based bioreactor. Further descriptions of suitable containers are contained herein. For illustrative purposes only, a suitable container can have a height of between about 10 and about 20 feet, particularly between about 12 and about 18 feet, and more particularly between about 14 and about 17 feet. For illustrative purposes only, a suitable container can have a diameter (at its widest cross-sectional portion) of between about 5 and about 15 feet, particularly between about 7 and about 13 feet, and more particularly between about 8 and about 12 feet. As illustrated in FIG. 9, in one embodiment, the kit includes a plurality of grating units 35 that combine to form a media support platform 30. As described above, the media support platform is permeable to liquids and is configured to be horizontally disposed inside the container. The kit also includes a cylindrical chamber, which includes an open top end, a bottom end, and a sidewall portion, where the bottom end is configured to be secured to the media support platform (as previously described herein). The kit further includes a plurality of mesh encasements, where each of the mesh encasements contains a plurality of media units. The kit also includes a recirculation/aeration system. The recirculation/aeration system includes a submersible pump device, an air intake tube, and an eductor device. The kit can further include a top cover for sealing the top port of the container. The kit can also further include a maintenance platform that is configured to be horizontally disposed inside the cylindrical chamber, where the maintenance platform is permeable to liquid and supports a weight of at least 200 pounds. The kit can also include a catch screen that is configured for attachment to the top end of the cylindrical chamber and is suitable for preventing solid material of a desired size from downwardly passing through the catch screen. The kit can be combined with a land-based container to form a bioreactor as described herein.

The present invention also relates to a method for converting a container into a bioreactor for treating a liquid. This method involves providing a container having a top portion, a vertical wall portion, and a bottom portion. In one embodiment, the top portion has at least a top port that includes an opening for access into the interior portion of the container. The top port can be sealable, and therefore can be provided either sealed or unsealed. Further, if the top portion does not include a top port, the top portion of the container can be configured to insert an opening therein, thereby allowing for access into the container from the top. The vertical wall portion of the container can have at least one inlet port and at least one outlet port. If the container does not include either of these two ports, the container can be configured to insert these ports. Suitable containers for use in this method can include any vertical container that can hold liquid. Therefore, the invention contemplates any non-liquid permeable vertical container of any size. Suitable containers can have a cross-section of any shape, including, but not limited to, a shape that is substantially a circle, a semicircle, a rectangle, a square, a triangle, a pentagon, a hexagon, a heptagon, an octagon, and/or an oval. Suitable materials comprising a suitable container include, without limitation, HDPE and the like, stainless steel and the like, or concrete.

Once the container is provided, a media support platform is inserted into the container. A suitable media support platform is one that is permeable to liquid. Suitable materials comprising the media support platform can include, without limitation, fiberglass, synthetic materials like fiberglass, and stainless steel and the like. This can be done by providing a plurality of media support panels that can be combined to form the media support platform. Therefore, each of the media support panels can be inserted through the top port of the container, and then assembled to combine the media support platform within the container. The media support platform is secured within the container at a position below the inlet and outlet ports of the container. In one embodiment, the media support platform is secured so that it is substantially perpendicular to the container's vertical wall portion and substantially spans the cross-sectional area of the container. The media support platform can be secured inside the container using any suitable means.

This method also involves depositing at least one cylindrical chamber inside the container at a position above the media support platform. A suitable cylindrical chamber is one that has a top end, a bottom end, and a sidewall portion. The top end is substantially open or completely open so that liquid can flow down through it. In one embodiment, a catch screen can be placed over the top end of the container, or the catch screen can be secured within the cylindrical chamber. The cylindrical chamber can be secured to the bottom portion of the container using support components. The cylindrical chamber can alternatively be positioned so that a portion of its bottom end extends below the media support platform. Suitable materials comprising the cylindrical chamber can include, without limitation, HDPE or the like. The top end can extend above the inlet and outlet ports of the container, at substantially the same level of the inlet and outlet ports, or below the inlet and outlet ports. Further, more than one cylindrical chamber can be deposited into the container in the same way as described above. In addition, a diverter for directing hydraulic flow of liquid exiting the eductor can be positioned below the eductor (i.e., in the bottom portion of the container at a position below the media support platform). A suitable diverter can be configured to be a 4-sided pyramid, or any configuration that will allow for even distribution of the liquid exiting the eductor, prior to the liquid's upward flow through the container.

This method further involves loading at least one flexible mesh encasement onto the media support platform in the outer chamber portion of the container. As described above, the outer chamber portion is defined by the space between the sidewall portion of the at least one cylindrical chamber and the vertical wall portion of the container. The at least one flexible mesh encasement contains a plurality of media units and has two opposed ends.

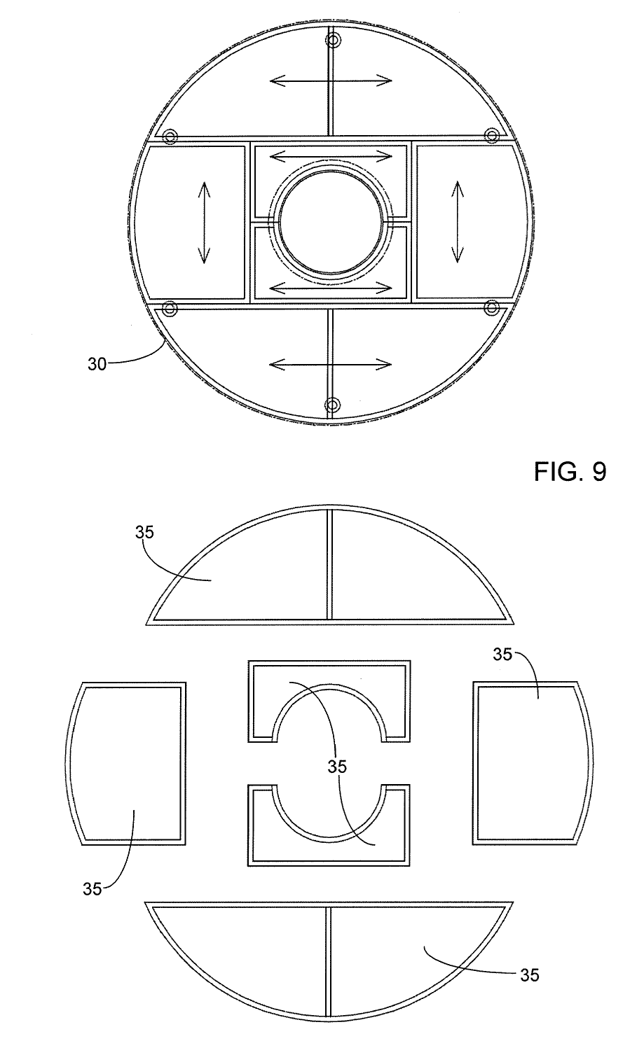

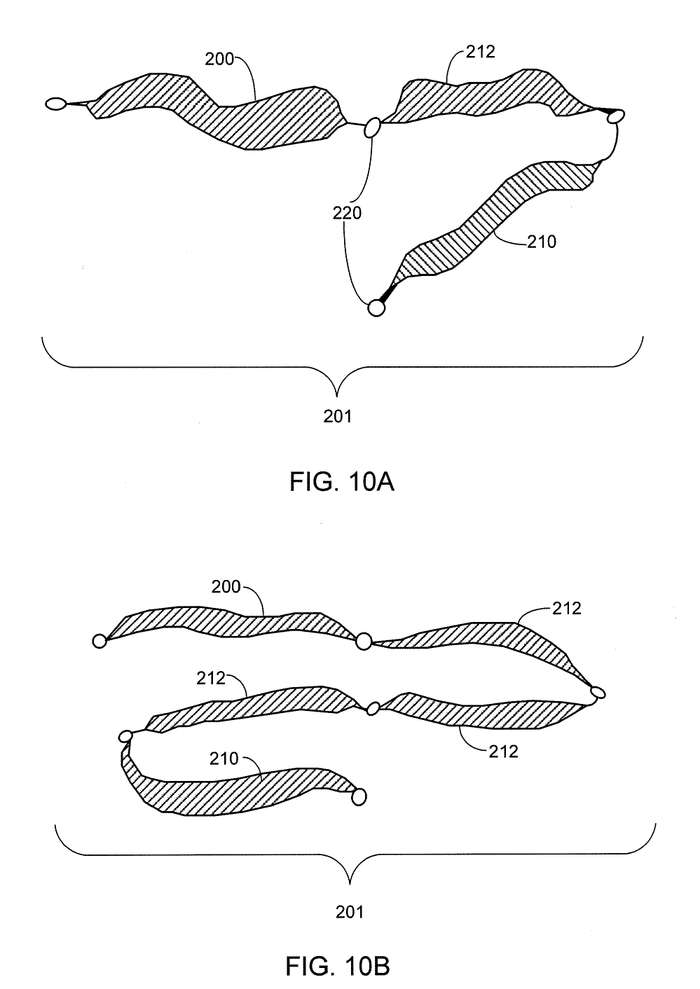

These opposed ends are closed to keep the media units from escaping from the encasements, and the ends may have hook/release devices (e.g., carabiner-type clips or hooks) attached thereto (see FIGS. 10A-B, described below). Loading the at least one flexible mesh encasement can include loading a plurality of flexible mesh encasements that each of are serially linked together from end to end (e.g., by attachment using the hook/release devices). When serially linked together, the plurality of flexible mesh encasements form a chain of encasements, so that the chain includes at least a first end encasement and at least an opposing second end encasement. In reference to FIGS. 10A-B, at least one internal encasement 212 can be attached between the first end encasement 200 and the second end encasement 210. In one embodiment, a maintenance platform is included in at least one of the cylindrical chambers so that a person can stand on the maintenance platform to facilitate loading (and unloading) of the flexible mesh encasements.

This method also involves installing a recirculation/aeration system (described previously) inside the cylindrical chamber. As described above, the recirculation/aeration system includes (i) a submersible pump device that can be programmed to pump liquid at variable speeds and that includes a liquid intake portion having perforations, (ii) an air intake tube, and (iii) an eductor device. Installation can be such that the air intake tube protrudes through the top port of the container's top portion.

The present invention further relates to a method of unloading media units from a container. Suitable containers includes those containers already mentioned above. This method involves identifying a container having a plurality of flexible mesh encasements, with each flexible mesh encasement containing a plurality of media units and having two opposed ends. As shown in FIGS. 10A-10B, the plurality of flexible mesh encasements can be serially linked together from end to end to form a chain 201 of encasements, where the chain includes at least a first end encasement 200 and an opposing second end encasement 210. The method further involves extracting the chain of encasements from the container, thereby unloading the plurality of media units from the container. In one embodiment, the plurality of flexible mesh encasements are serially linked together by hook/release devices 220 (described above) attached at each end of each flexible mesh encasement.

In one embodiment of this method, the extracting step includes selecting the first end encasement of the chain and thereafter unloading the chain by serially exporting it from the container. In reference to FIGS. 10A-B, in this embodiment, it is contemplated that the first end encasement 200 is exported from the container first, while the second end encasement 210 is exported last. In another embodiment, the extracting step includes (a) unlinking at least one serially linked encasement from the chain; (b) exporting the unlinked encasement from the container; and (c) repeating steps (a) and (b) until all of the encasements are exported from the container. In yet another embodiment, the extracting step includes (a) unlinking at least one serially linked encasement from the chain, where the unlinking yields a chain of at least two serially linked encasements; (b) exporting the at least two serially linked encasement from the container; and (c) repeating steps (a) and (b) until all of the encasements are exported from the container.

The advantages of the vertical land-based bioreactor of the present invention, as well as the system including the bioreactor and methods of using the bioreactor to treat liquids have been discussed herein. Embodiments discussed have been described by way of example in this specification. It will be apparent to those skilled in the art that the foregoing detailed disclosure is intended to be presented by way of example only, and is not limiting. Various alterations, improvements, and modifications will occur and are intended to those skilled in the art, though not expressly stated herein. These alterations, improvements, and modifications are intended to be suggested hereby, and are within the spirit and the scope of the claimed invention. Additionally, the recited order of processing elements or sequences, or the use of numbers, letters, or other designations therefore, is not intended to limit the claims to any order, except as may be specified in the claims. Accordingly, the invention is limited only by the following claims and equivalents thereto.

What is claimed is:

1. A bioreactor for treating a liquid, said bioreactor comprising:
a container comprising a top portion, a vertical wall portion, and a bottom portion, wherein said top portion comprises at least a top port that is either sealed or unsealed, and wherein said vertical wall portion comprises at least one inlet port and at least one outlet port;
a media support platform securely disposed inside the container at a position below the inlet and outlet ports, wherein the media support platform is substantially perpendicular to the container's vertical wall portion and substantially spans the cross-sectional area of the container, and wherein the media support platform is permeable to liquid;
at least one cylindrical chamber securely disposed inside the container at a position above the media support platform, wherein said at least one cylindrical chamber comprises a top end, a bottom end, and a sidewall portion;
a plurality of media units topically disposed on the media support platform in an outer chamber portion, wherein said outer chamber portion is defined by the space between the sidewall portion of the at least one cylindrical chamber and the vertical wall portion of the container; and
a recirculation/aeration system secured at the bottom end of the cylindrical chamber, wherein said recirculation/aeration system comprises a submersible pump device that can be programmed to pump liquid at variable speeds and that comprises a liquid intake portion having perforations, an air intake tube, and an eductor device, and wherein the air intake tube protrudes through the top port of the container's top portion.

2. The bioreactor according to claim 1 further comprising a top cover for sealing the top port.

3. The bioreactor according to claim 2, wherein said top cover comprises an opening through which the air intake tube extends.

4. The bioreactor according to claim 3, wherein said top cover further comprises at least one opening for releasing gas from the container or for introducing nutrients and/or microorganisms into the container.

5. The bioreactor according to claim 1, wherein said media support platform comprises a plurality of media support panels.

6. The bioreactor according to claim 1, wherein said cylindrical chamber further comprises a maintenance platform horizontally secured inside the cylindrical chamber at a position above the submersible pump device, wherein said maintenance platform is permeable to liquid and configured to support a weight of at least 200 pounds.

7. The bioreactor according to claim 1 further comprising a catch screen horizontally secured inside the cylindrical chamber at a position above the submersible pump device, wherein said catch screen comprises openings that are permeable to liquid but not permeable to solid materials of a size larger than the perforations of the liquid intake portion of the submersible pump device.

8. The bioreactor according to claim 1 further comprising a baffle for directing flow of surface liquid within the container.

9. The bioreactor according to claim 8, wherein the baffle is positioned at the top end of the cylindrical chamber to promote flow of surface liquid into the top end of the cylindrical chamber.

10. The bioreactor according to claim 8, wherein the baffle is configured to inhibit surface liquid from entering the inlet port and passing directly out of the outlet port.

11. The bioreactor according to claim 1, wherein said plurality of media units are contained in a plurality of mesh encasements.

12. The bioreactor according to claim 1 further comprising an air supply device located below the media support platform, wherein said air supply device is configured to introduce air into the container.

13. The bioreactor according to claim 12, wherein the air is introduced coarse bubbles.

14. The bioreactor according to claim 1, wherein said liquid is wastewater.

15. A method for treating a liquid, said method comprising:
providing a bioreactor according to claim 1;
importing an untreated liquid into the bioreactor;
recirculating the untreated liquid within the bioreactor to produce a treated liquid; and
exporting the treated liquid from the bioreactor.

16. The method according to claim 15, wherein importing the untreated liquid into the bioreactor comprises pumping or gravity-feeding the untreated liquid into an inlet port of the bioreactor.

17. The method according to claim 15, wherein recirculating the untreated liquid within the bioreactor comprises flowing the untreated liquid through at least one flow cycle, wherein one flow cycle comprises:
running the untreated liquid through the recirculation/aeration system to produce aerated untreated liquid and
directing the aerated untreated liquid upward through the media units to produce a treated liquid.

18. The method according to claim 15, wherein recirculating the untreated liquid within the bioreactor comprises flowing the untreated liquid through a plurality of flow cycles prior to exporting the treated liquid from the bioreactor.

19. The method according to claim 15, wherein the top port of the bioreactor is sealed with a top cover.

20. The method according to claim 19, wherein the top cover comprises at least one opening for manually releasing gas from the bioreactor.

21. The method according to claim 15 further comprising introducing at least one type of microorganism into the bioreactor and contacting the at least one type of microorganism to the media units contained in the bioreactor to promote biofilm growth on the media units.

22. The method according to claim 15 further comprising introducing air into the container at a position below the media support platform to inhibit, wherein said air is introduced as either coarse bubbles or fine bubbles.

23. The method according to claim 22, wherein said introducing promotes coarse bubble diffusion upwardly through the media units.

24. The method according to claim 15, wherein said liquid is wastewater.

25. A system for treating a liquid, said system comprising:
a source of untreated liquid;
at least one bioreactor according to claim 1, wherein the at least one bioreactor is coupled to the source of untreated liquid and configured to receive untreated liquid from the source and to treat the untreated liquid to produce treated liquid; and
a collection unit for collecting the treated liquid from the at least one bioreactor, wherein the collection unit is coupled to the at least one bioreactor, and wherein said collection unit is configured to clarify and/or agitate the collected treated liquid.

26. The system according to claim 25 further comprising an external pump for importing the untreated liquid from the source into the at least one bioreactor.

27. The system according to claim 25, wherein the top port of the at least one bioreactor is sealed with a top cover to prevent release of gas from the at least one bioreactor.

28. The system according to claim 27, wherein the top cover comprises at least one opening for manually releasing gas from the at least one bioreactor.

29. The system according to claim 25 further comprising an air supply device disposed below the media support platform of the at least one bioreactor, wherein said air supply device is configured to introduce air into the container as either coarse bubbles or fine bubbles.

30. The system according to claim 25, wherein the air supply device is configured to promote coarse bubble diffusion upwardly through the media units of the container.

31. The system according to claim 25, wherein said liquid is wastewater.

32. The system according to claim 25, wherein the at least one bioreactor comprises a plurality of serially linked bioreactors .

33. A kit for converting a container into a bioreactor for treating a liquid, said kit comprising:
a plurality of media support panels that combine to form a media support platform inside a container, wherein the media support platform is permeable to liquid;
at least one cylindrical chamber comprising a top end, a bottom end, and a sidewall portion, wherein the bottom end is configured to be secured inside the container;
a plurality of media units contained in at least one flexible mesh encasement; and
a recirculation/aeration system comprising a submersible pump device that can be programmed to pump liquid at variable speeds and that comprises a liquid intake portion having perforations, an air intake tube, and an eductor device.

34. The kit according to claim 33 further comprising a top cover for sealing the top port of the container.

35. The kit according to claim 33 further comprising a maintenance platform that is configured to be horizontally secured inside the cylindrical chamber, wherein said maintenance platform is permeable to liquid and supports a weight of at least 200 pounds.

36. The kit according to claim 33 further comprising a catch screen that is configured to be horizontally secured inside the cylindrical chamber, wherein said catch screen is permeable to liquid but not permeable to solid materials of a size larger than the perforations of the liquid intake portion of the submersible pump device.

37. A method for converting a container into a bioreactor for treating a liquid, said method comprising:
providing a container, wherein said container comprises a top portion, a vertical wall portion, and a bottom portion, wherein said top portion comprises at least a top port that is either sealed or unsealed, and wherein said vertical wall portion comprises at least one inlet port and at least one outlet port;
inserting a media support platform inside the container at a position below the inlet and outlet ports, wherein the media support platform is substantially perpendicular to the container's vertical wall portion and substantially spans the cross-sectional area of the container, and wherein the media support platform is permeable to liquid;
depositing at least one cylindrical chamber inside the container at a position above the media support platform, wherein said at least one cylindrical chamber comprises a top end, a bottom end, and a sidewall portion;
loading at least one flexible mesh encasement onto the media support platform in an outer chamber portion of the container, wherein said outer chamber portion is defined by the space between the sidewall portion of the at least one cylindrical chamber and the vertical wall portion of the container, and wherein the at least one flexible mesh encasement contains a plurality of media units; and
installing a recirculation/aeration system inside the cylindrical chamber, wherein said recirculation/aeration system comprises a submersible pump device that can be programmed to pump liquid at variable speeds and that comprises a liquid intake portion having perforations, an air intake tube, and an eductor device, and wherein the air intake tube protrudes through the top port of the container's top portion.

38. The method according to claim 37, wherein loading the at least one flexible mesh encasement comprises loading a plurality of flexible mesh encasements that are serially linked together from end to end.

39. A method of unloading media units from a container, said method comprising:
identifying a container having a plurality of flexible mesh encasements each containing a plurality of media units and each having two opposed ends, wherein said plurality of flexible mesh encasements are serially linked together from end to end to form a chain of encasements, wherein said chain comprises a first end encasement and an opposing second end encasement; and
extracting the chain of encasements from the container, thereby unloading the plurality of media units from the container.

40. The method according to claim 39, wherein the plurality of flexible mesh encasements are serially linked together by hook/release devices attached at each end of each flexible mesh encasement.

41. The method according to claim 39, wherein said extracting step comprises:
selecting the first end encasement and
unloading the chain of encasements by serially exporting the chain from the container, wherein the first end encasement is exported first and the second end encasement is exported last.

42. The method according to claim 39, wherein said extracting step comprises:
(a) unlinking at least one serially linked encasement from the chain;
(b) exporting the unlinked encasement from the container; and
(c) repeating steps (a) and (b) until all of the encasements are exported from the container.

43. The method according to claim 39, wherein said extracting step comprises:
(a) unlinking at least one serially linked encasement from the chain, wherein said unlinking yields a chain of at least two serially linked encasements;
(b) exporting the at least two serially linked encasement from the container; and
(c) repeating steps (a) and (b) until all of the encasements are exported from the container.

44. The method according to claim 39, wherein the container comprises a bioreactor used for treating liquids.
